(19) United States
(12) Patent Application Publication
(10) Pub. No.: US 2006/0159718 A1
(43) Pub. Date: Jul. 20, 2006

(54) Method for the production of porous carbon-based molded bodies, and use thereof as cell culture carrier systems and culture systems

(76) Inventors: Jorg Rathenow, Eppstein (DE); Soheil Asgari, Wiesbaden (DE); Jurgen Kunstmann, Bad Soden (DE)

Correspondence Address: DORSEY & WHITNEY LLP;INTELLECTUAL PROPERTY DEPARTMENT, 250 PARK AVENUE, NEW YORK, NY, 10177, US

(21) Appl. No.: 11/343,480

(22) Filed: Jan. 30, 2006

Related U.S. Application Data
(63) Continuation-in-part of application No. PCT/EP04/00077, filed on Jan. 8, 2004.

(30) Foreign Application Priority Data
Jul. 31, 2003 (DE) DE 103 35 131.0

(51) Int. Cl. A61F 2/28; C01B 31/00; A61F 13/00

(52) U.S. Cl. 424/422; 264/29.1; 623/16.11

(57) ABSTRACT

The present invention relates to methods for producing carbon-based molded bodies. In particular, the present invention relates to methods for producing porous carbon-based molded bodies by carbonizing organic polymer materials mixed with non-polymeric fillers and subsequently dissolving the fillers out from the carbonized molded bodies. The present invention further relates to methods for producing porous carbon-based molded bodies by carbonizing organic polymer materials mixed with non-polymeric fillers which are substantially completely decomposed during the carbonization. The present invention also relates to a method for producing porous carbon-based molded bodies by carbonizing organic polymer materials, the carbon-based molded bodies being partially oxidized following carbonization so as to produce pores. In addition, the present invention relates to porous molded bodies produced according to one of said methods and the use thereof, especially as cell culture carriers and/or culture systems.

### CROSS-REFERENCE TO RELATED APPLICATION(S)

This application is a continuation-in-part application of International Patent Application No. PCT/EP2004/000077, filed Jan. 8, 2004, which claims priority from German Patent Application No. DE 103 35 131.0, filed Jul. 31, 2003, the entire disclosures of which are incorporated herein by reference.

### BACKGROUND OF THE INVENTION

As a result of the variability of its properties, carbon is a versatile material in many areas of materials engineering. Carbon-based materials may be used in mechanical engineering applications, vehicle construction, and also in medical engineering and process engineering applications. German Patent Application No. DE 35 28 185 describes a method for producing high-strength, high-density carbon materials from special powdered carbon-containing raw materials without using a binder.

German Patent Application No. DE 198 23 507 describes methods for producing carbon-based shaped bodies by carbonizing biogenic raw materials of natural vegetable fibers or wood product. German Patent Application No. DE 100 11 013 and European Patent Application No. EP 0 543 752 describe methods for producing carbon-containing materials by carbonization or pyrolysis of foamed initial polymers such as polyacrylnitrile or polyurethane. The carbon foams thus obtained can be used as high-temperature insulators in furnace installations or reactor construction, or for sound dampening in high-temperature operations. U.S. Pat. No. 3,342,555 also describes a method for producing light porous carbon by carbonizing foamed polymers based on phenolaldehyde resins of the resol or novolac type.

Conventional methods for producing porous carbon molded bodies have the disadvantage that the molded bodies obtained by carbonizing foamed polymers may frequently exhibit very low mechanical stability, which can makes it difficult to use these under mechanical loading conditions. Further, it may not be possible to adjust or control the pore size and/or pore volume in these molded bodies accurately enough for these to be usable, for example, in biotechnological applications such as orthopedic implants.

There is thus a need for new and improved methods for producing porous carbon-containing molded bodies.

### SUMMARY OF THE INVENTION

Exemplary embodiments of the present invention relate to methods for producing carbon-based molded bodies. In particular, the exemplary embodiments of the present invention relate to methods for producing porous carbon-based molded bodies by carbonizing organic polymer materials mixed with non-polymeric fillers and subsequently dissolving the fillers out from the carbonized molded bodies. The exemplary embodiments of the present invention further relate to methods for producing porous carbon-based molded bodies by carbonizing organic polymer materials mixed with non-polymeric fillers which are substantially completely decomposed during the carbonization. The exemplary embodiments of the present invention also relates to a method for producing porous carbon-based molded bodies by carbonizing organic polymer materials, the carbon-based molded bodies being partially oxidized following carbonization so as to produce pores. In addition, the exemplary embodiments of the present invention further relates to porous molded bodies produced according to one of said methods and the use thereof, especially as cell culture carriers and/or culture systems.

It is thus one of the objects of the present invention to provide a method for producing porous carbon-based molded bodies which can be economically manufactured and implemented.

Another of the objects of the present invention is to provide a method for producing porous carbon-based molded bodies which allows the porosity, particularly the pore volume and the pore diameter, to be adjusted in a reproducible manner by varying process parameters.

Yet another object of the present invention is to provide methods for producing porous carbon-based molded bodies in a variety of shapes and dimensions.

It is a further object of the present invention to provide fields of use and applications for the porous carbon-based molded bodies.

It is still another object of the present invention to provide methods, whereby porous carbon-based molded bodies can be produced by carbonizing semi-finished molded parts of organic polymer materials, where the porosity of the molded body may be produced during or following the pyrolysis.

These and other objects, features and advantages of the present invention will become apparent upon reading the following detailed description of embodiments of the invention, when taken in conjunction with the appended claims.

### DETAILED DESCRIPTION OF EXEMPLARY EMBODIMENTS

In a first exemplary embodiment of the present invention, a method is provided for producing porous carbon-based molded bodies comprising:
(a) mixing organic polymer materials, which can be carbonized to form carbon, with non-polymeric fillers;
(b) producing a semi-finished molded part from the mixture;
(c) carbonizing the semi-finished molded part in a non-oxidizing atmosphere at elevated temperatures, whereby a carbon-based molded body is obtained; and
(d) dissolving the fillers contained in the carbonized molded body using suitable solvents.
   In this exemplary embodiment of the present invention, the organic polymer materials in (a) above can be mixed or blended with non-polymeric fillers. This can be carried out using suitable conventional mixing methods such as, for example, dry mixing of polymer pellets with filler powders or granules, mixing fillers into the polymer melt, or mixing fillers with polymer solutions or suspensions.

Suitable non-polymeric fillers can include substances which are substantially stable under carbonization conditions and which can be removed from the carbon-based molded bodies after carbonization by using suitable solvents. Furthermore, non-polymeric fillers which are converted to solvent-soluble substances under carbonization conditions may also be suitable as fillers.

Preferred fillers can include, but are not limited to, inorganic metal salts, especially salts of alkali and/or alkaline earth carbonates, sulphates, sulphites, nitrates, nitrites, phosphates, phosphites, halides, sulphides, oxides, and mixtures thereof. Further suitable fillers include organic metal salts, preferably those of alkali, alkaline-earth and/or transition metals, especially their formates, acetates, propionates, maleates, malates, oxalates, tartrates, citrates, benzoates, salicylates, phthalates, stearates, phenolates, sulphonates, amine salts, and mixtures thereof.

Suitable solvents for dissolving out the fillers from the carbonized molded bodies can include water, especially hot water, diluted or concentrated inorganic or organic acids, alkalis and the like. Suitable inorganic acids include, in diluted or concentrated form, hydrochloric acid, sulphuric acid, phosphoric acid, nitric acid, or diluted hydrofluoric acid.

Suitable alkalis can include, for example, sodium hydroxide solution, ammonia solution, carbonate solutions, or organic amine solutions.

Suitable organic acids can include formic acid, acetic acid, trichloromethanoic acid, trifluoromethanoic acid, citric acid, tartaric acid, oxalic acid, and mixtures thereof.

The fillers can be partially or substantially completely dissolved out from the carbonized molded body, according to the type and duration of usage of the solvent. Substantially complete dissolution of the fillers may be performed.

The fillers can be provided in suitable grain sizes depending on the intended application and desired porosity or pore dimension. Powder or granular fillers may have an average particle size of about 3 Å to 2 mm, or preferably about 1 nm to 500 μm, or more preferably about 10 nm to 1100 μm.

Suitable particle sizes of non-polymeric fillers may be selected based on the desired porosity and the desired pore dimensions of the ready-carbonized molded body.

In addition, suitable solvents for dissolving out the fillers may include organic solvents such as methanol, ethanol, N-propanol, isopropanol, butoxydiglycol, butoxyethanol, butoxyisopropanol, butoxypropanol, n-butyl alcohol, t-butyl alcohol, butylene glycol, butyl octanol, diethylene glycol, dimethoxydiglycol, dimethylether, dipropylene glycol, ethoxydiglycol, ethoxyethanol, ethyl hexanediol, glycol, hexanediol, 1,2,6-hexanetriol, hexylalcohol, hexylene glycol, isobutoxypropanol, isopentyl diol, 3-methoxybutanol, methoxydiglycol, methoxyethanol, methoxyisopropanol, methoxymethylbutanol, polypropylene glycol, methylal, methyl hexylether, methylpropanediol, neopentyl glycol, polyethylene glycol, pentylene glycol, propanediol, propylene glycol, propylene glycol butylether, propylene glycol propylether, tetrahydrofuran, trimethylhexanol, phenol, benzene, toluol, xylol, and water, which may be optionally mixed with dispersion adjuvants, as well as mixtures of the aforesaid.

In certain exemplary embodiments of the present invention, mixtures of organic solvents with water and/or inorganic and/or organic acids can also be used to dissolve out the non-polymeric fillers from the carbonized molded bodies.

In a further exemplary embodiment of the present invention, a method for producing porous carbon-based molded bodies can be provided, comprising:
(a) mixing organic polymer materials, which can be carbonized to form carbon, with polymeric fillers;
(b) producing a semi-finished molded part from the mixture; and
(c) carbonizing the semi-finished molded part in a non-oxidizing atmosphere at elevated temperatures, wherein the polymeric fillers are substantially completely decomposed.

The pores in the carbon-based molded body may be produced during carbonization from the polymeric fillers that are incorporated in the organic polymer materials to be carbonized, where the polymeric fillers can be substantially decomposed under carbonization conditions.

Certain polymeric fillers, especially saturated aliphatic hydrocarbons, can be decomposed substantially completely under carbonization conditions, i.e. high temperatures and in the absence of oxygen, by using methods similar to cracking to yield volatile hydrocarbons such as methane, ethane and the like, which then escape from the porous carbon framework of the carbonized molded body during pyrolysis or carbonization.

Suitable polymeric fillers can include saturated, branched or unbranched aliphatic hydrocarbons, which can be homo- or copolymers. It may be preferable to use polyolefins such as polyethylene, polypropylene, polybutene, polyisobutene, polypentene as well as their copolymers and mixtures thereof.

Initially, the polymeric fillers can be mixed with the carbonizable polymer materials. This can be carried out using suitable conventional mixing methods such as, for example, mixing of polymer pellets or granules, mixing polymeric fillers into melts of carbonizable organic polymer materials or suspensions or solutions of these polymer materials, coextrusion of the polymeric fillers with the carbonizable organic polymer materials, and the like.

The pores produced in the carbonized molded bodies can be suitably dimensioned or varied within wide limits by a suitable choice of molecular weight, chain length and/or degree of branching of the polymeric fillers. The polymeric fillers can also be used in the form of thin fibers which may form suitably dimensioned pore passages during carbonization. The porosity can be adjusted by selecting the fiber diameter and the fiber length, whereby larger fiber diameters and/or lengths may produce greater porosity. Other properties, including asymmetrical porosity distributions and textures of the molded bodies, can also be achieved by suitable mixing of fibers having different properties.

This exemplary embodiment of the method of the present invention, which includes using polymeric fillers to form pores, may be suitable for producing porous molded bodies having small pore sizes in the nano- to micrometer range, especially those having pore sizes in the range of about 3 Å to 2 mm, or preferably about 1 nm to 500 μm, or more preferably about 10 nm to 100 μm.

In a further exemplary embodiment of the methods of the present invention, the carbonized molded body may be treated after carbonization with suitable oxidizing and/or reducing agents to further modify the pore sizes. A subsequent compaction or closure of the pores, for example by CVD or CVI methods, while separating suitable organic or inorganic precursors, can also be used to tailor molded bodies having desired properties.

A further exemplary embodiment of the present invention provides a method for producing porous carbon-based molded bodies, comprising:
(a) producing a semi-finished molded part from carbonizable organic polymer materials;
(b) carbonizing the semi-finished molded part in a non-oxidizing atmosphere at elevated temperatures, whereby a carbon-based molded body is obtained; and
(c) partially oxidizing the carbonized molded body to produce pores.

In accordance with this exemplary embodiment, a molded body may be formed by carbonizing suitable polymer materials. After carbonization, porosity can be produced and/or enlarged or enhanced in the carbonized molded body by means of suitable oxidizing agents, by “burning” pores into the carbon-based molded bodies through partial oxidation of the carbon.

The treatment of the carbonized molded body can take place at elevated temperatures in oxidizing gas atmospheres. Suitable oxidizing agents for partial oxidation in an oxidizing gas phase include, but are not limited to, air, oxygen, carbon monoxide, carbon dioxide, nitrogen oxide, and similar oxidizing agents. These gaseous oxidizing agents can be mixed with inert gases such as noble gases, including argon or nitrogen, which may allow for adjustment of suitable volume concentrations of the oxidizing agent. Holes or pores can be burned into the porous molded body by reaction with these oxidizing agents through partial oxidation.

The partial oxidation can be carried out at elevated temperatures, particularly in the range of about 50° C. to 800° C.

In an exemplary embodiment of the present invention, partial oxidation may be carried out by treating the molded body with air at room temperature or elevated temperatures, where the air may optionally be flowing.

In addition to the partial oxidation of the molded body by gaseous oxidizing agents, liquid oxidizing agents can also be used, such as, for example, concentrated nitric acid, which may be applied to the molded body in a suitable manner. In this exemplary embodiment, the concentrated nitric acid can be brought into contact with the carbonized molded body at temperatures above room temperature to create superficial or deeper pore formation.

The aforementioned exemplary methods of the present invention for producing pores can also be combined with one another. Thus, both soluble fillers and polymeric fillers, which can be volatile under carbonization conditions or can be decomposed to give volatile substances, may be used to form a single porous molded body. Coarser pores produced by the soluble fillers can be linked to the micropores or nanopores produced by the polymeric fillers to yield anisotropic and/or multiscale pore distributions. Existing pores can also be expanded, interlinked, or modified by partial oxidation of the molded body.

In further exemplary embodiments of the present invention, pores may also be closed, for example, by treatment with liquid-crystal tar pitch and then optionally subjected to renewed temperature treatment. High-ordered crystalline zones can be achieved by carbonization in this manner. Asymmetric and symmetrical graded materials, for example, can be obtained by various combinations of the exemplary methods described above.

Exemplary Organic Polymer Material

In exemplary embodiments of the present invention such as those described above, the organic polymer materials which may be used can include those which can be carbonized to form carbon-based materials that may be amorphous, partially crystalline and/or crystalline, and which may further be symmetrical or asymmetrical, under carbonization conditions, i.e., at elevated temperature and in a substantially oxygen-free atmosphere.

Unsaturated, branched aliphatic hydrocarbons, branched or unbranched cross-linked or non-cross-linked aromatic or partially aromatic hydrocarbons, and substituted derivatives thereof can be suitable organic polymer materials. Unsaturated hydrocarbons, including aromatic hydrocarbons, can be converted into graphite-like cross-linked six-ring structures under carbonization conditions, which can form the basic framework of the resulting carbonized molded body.

Saturated aliphatic and/or aromatic hydrocarbons with heteroatom fractions, such as ether, urethanes, amides, amines and the like, may also be suitable as carbonizable organic polymer materials, either by themselves or when mixed with other aliphatic or aromatic unsaturated hydrocarbons.

The carbonizable organic polymer materials can include, but are not limited to, polybutadiene; polyvinyls such as polyvinylchloride or polyvinyl alcohol, poly(meth)acrylic acid, polyacryl cyanoacrylate; polyacrylnitrile, polyamide, polyester, polyurethane, polystyrene, polytetrafluoroethylene; polymers such as collagen, albumin, gelatin, hyaluronic acid, starch, celluloses such as methylcellulose, hydroxypropylmethyl cellulose, carboxymethyl cellulose phthalate; casein, dextran, polysaccharide fibrinogen, poly(D,L-lactide), poly(D,L-lactide-co-glycolide), polyglycolide, polyhydroxybutylate, polyalkylcarbonate, polyorthoester, polyester, polyhydroxyvaleric acid, polydioxanone, polyethylene terephthalate, polymalic acid, polytartaric acid, polyanhydride, polyphosphazene, polyamino acids; polyethylenevinyl acetate, silicone; poly(ester urethane), poly(ether urethane), poly(ester urea), polyether such as polyethylene oxide, polypropylene oxide, pluronics, polytetramethylene glycol; polyvinyl pyrrolidone, poly(vinyl acetate phthalate), alkyd resin, chlororubber, epoxy resin, acrylate resin, phenol resin, amine resin, melamine resin, alkylphenol resins, epoxided aromatic resins, tar, tar-like materials, tar pitch, liquid-crystal tar pitch, bitumen, starch, cellulose, shellac, organic materials of renewable raw materials, as well as copolymers, mixtures or combinations of these homo- or copolymers.

The carbonizable polymer materials can also contain additives such as fillers, softeners, lubricants, flame retardants, glass, glass fibers, carbon fibers, cotton, fabric, metal powder, metal compounds, metal oxides, silicon, silicon oxide, zeolites, titanium oxide, zirconium oxide, aluminium oxide, aluminosilicate, talc, graphite, soot, clay materials, phyllosilicates and the like. Fibrous materials such as cellulose, cotton, textile fabrics, glass fibers, carbon fibers and the like may be preferable as polymer additives for improving the mechanical properties of the porous molded bodies produced.

Semi-finished molded bodies can be produced by shaping methods conventionally used for polymer materials. Suitable shaping methods include, but are not limited to, casting methods, extrusion methods, pressing methods, injection molding methods, co-extrusion blow molding, or other shaping methods, for example, winding methods or strand winding methods that may be applied to flat starting materials.

Exemplary Carbonization

In certain exemplary embodiments of the present invention, carbonization can be carried out in a substantially oxygen-free or oxidizing-agent-free atmosphere. Suitable carbonizing atmospheres include, for example, protective or inert gases such as nitrogen and/or argon, SiF₆, or mixtures of these gases. Optionally, these protective or inert gas atmospheres can be present at pressures below or above atmospheric pressure. Carbonization can also be performed under a vacuum.

In further exemplary embodiments of the present invention, reactive gases can be added to the protective or inert gas atmospheres. Reactive gases that may be used for this purpose include non-oxidizing gases such as hydrogen, ammonia, C₁-C₆ saturated aliphatic hydrocarbons such as methane, ethane, propane or butane, mixtures of these gases, and the like.

Suitable temperatures for the carbonization step can be in the range of about 200 C to 4000 C or more. Depending on the selected temperature in the carbonization step and the type of polymer material used, carbon-containing molded bodies can be produced where the base material may have a structure ranging from amorphous to ordered crystalline graphite-like structures, or a mixtures of both structures.

Suitable temperature, atmosphere and pressure conditions can be selected based on the temperature-dependent properties of the specific polymer materials used and/or the starting material mixtures, which may include polymeric and/or non-polymeric fillers.

The atmosphere provided for the carbonization step may be substantially free from oxygen, having O₂ concentrations below about 10 ppm, or preferably below about 1 ppm. It is preferable to use hydrogen or inert gas atmospheres comprising, for example, nitrogen, argon, neon, or other inert gases which do not react significantly with carbon, or gas compounds and mixtures thereof.

The exemplary carbonization step can be performed in a discontinuous method in suitable furnaces, or optionally in continuous furnace processes.

In a continuous furnace process, the semi-finished molded parts can be introduced into the furnace on one side and be withdrawn from the other end of the furnace. In certain exemplary embodiments of the present invention, a semi-finished molded part can be placed in the furnace on a perforated plate, a sieve or the like, so that essentially the entire surface of the polymer body or film can be exposed to low pressure during the pyrolysis or carbonization. This exemplary embodiment can allow the implants to be fixed in the furnace, and also may permit improved extraction and better flow of inert gases through the semi-finished molded parts during the carbonization.

The furnace can be divided into separate regions by the use of inert-gas locks, in which either one or a plurality of carbonization steps can be carried out successively, optionally under different carbonization conditions in the different regions. The different regions may comprise, for example, different temperatures, different inert gases, and/or different pressures including a vacuum. Furthermore, optional after-treatment, activation, or intermediate treatment steps can be carried out in different regions of the furnace, such as, for example, partial oxidation, reduction, or impregnation with metal salt solutions and the like.

Alternatively, the carbonization can be carried out in a closed furnace, which may be preferred if the carbonization is to be carried out in a vacuum or under very low pressure. Depending on the carbonizable or organic polymer material or fillers used, the weight of the material can be reduced during the carbonization by about 5% to 95%, or preferably by about 40% to 90%, or more preferably by about 50% to 70%.

Exemplary After-Treatment

In certain exemplary embodiments of the present invention, the physical and chemical properties of the carbon-based molded bodies and/or the pores produced may be further modified after carbonization by suitable after-treatment steps that can be chosen based on their intended use.

Suitable after-treatments may include, for example, reductive or oxidative after-treatment steps in which the porous molded bodies can be treated with suitable reducing agents and/or oxidizing agents such as hydrogen, carbon dioxide, nitrogen oxides such as N₂O, water vapour, oxygen, air, nitric acid and the like, or mixtures thereof.

Furthermore, the surfaces of the molded bodies may be provided with coatings, which can be applied to one side or to both sides. Suitable coating materials can include, for example, the aforesaid organic polymer materials which may optionally be subjected to a further carbonization or pyrolysis after application to produce asymmetric textures in the molded body. Inorganic substances and biocompatible polymers and materials may also be used as coating materials to give the surfaces of the molded bodies certain desired properties.

The exemplary after-treatment procedures can optionally be carried out at elevated temperatures, which may be below the carbonization temperatures. After-treatment temperatures can be, for example, about 15 to 1000° C., preferably about 70 to 900° C., more preferably about 100 to 850° C., even more preferably about 200 to 800° C., or yet more preferably about 700° C. In certain exemplary embodiments of the present invention, the porous molded bodies produced may be modified reductively or oxidatively, or by using a combination of these after-treatment steps, optionally at room temperature.

The pore dimensions and their properties in the porous molded bodies can be specifically influenced or varied by oxidative or reductive treatment or by the incorporation of additives, fillers or functional materials. For example, the surface properties of the carbon-containing material can be hydrophilised or hydrophobised by incorporating inorganic nanoparticles or nanocomposites such as laminated silicates.

Furthermore, the porous molded bodies can be sealed on one or both sides by subsequent coating, e.g. with polymer solutions. This coating can optionally be subjected to an additional carbonization procedure, for example, to improve the stability.

The porous molded bodies can also be provided with biocompatible outer and/or inner surfaces by incorporating suitable additives. Molded bodies thus modified can be used, for example, as bioreactors, cell culture carrier systems or culture systems, implants, or as pharmaceutical carriers or depots, including systems which can be implanted into the body. In the latter case, for example, medicaments or enzymes can be incorporated into the material where these can optionally be released in a controlled fashion by suitable retardation and/or selective permeation properties of applied coatings.

The porous molded body can optionally also be subjected to a CVD process (Chemical Vapor Deposition) or CVI process (Chemical Vapor Infiltration) in order to further modify the surface or pore structure and its properties, or optionally to superficially or completely seal the pores. For this purpose, the carbonized coating may be treated with suitable carbon-separating precursor gases at high temperatures. Other elements can also be separated in this way, for example, silicon, aluminum or titanium, optionally to produce the corresponding carbides. By suitably pre-structuring the molded bodies, for example, by using fiber materials of different length and/or thickness, graded materials can thus be obtained. Such graded materials may have asymmetric concentration distributions of certain interstitial or reaction compounds, for example, of metal or non-metal carbides, nitrides or borides, through the volume of the molded body. Graded materials thus formed may be provided with symmetrical or asymmetrical, isotropic or anisotropic, closed-pore, porous or fiber-like guide structures, or any combinations thereof.

Saturated and unsaturated hydrocarbons having sufficient volatility under CVD conditions can be used as carbon-separating precursors. Examples of these can include methane, ethane, ethylene, acetylene, linear and branched alkanes, alkenes and alkynes with carbon numbers C₁-C₂₀, aromatic hydrocarbons such as benzene, naphthalene and the like, as well as singly and multiply alkyl-, alkenyl- and alkynyl-substituted aromatic compounds such as toluol, xylol, cresol, styrene, and similar compounds.

Compositions such as BCl₃, NH₃, silanes such as SiH₄, tetraethoxysilane (TEOS), dichlorodimethylsilane (DDS), methyltrichlorosilane (MTS), trichlorosilyldichloroborane (TDADB), hexadichloromethylsilyloxide (HDMSO), AlCl₃, TiCl₃ or mixtures thereof, may be used as ceramic precursors. These precursors may be used in the CVD processes described above in low concentrations of about 0.5 to 15 vol. % when mixed with an inert gas such as, for example, nitrogen, argon or the like. It is also possible to add hydrogen to certain separating gas mixtures. These compounds can separate hydrocarbon fragments or carbon or ceramic precursors which may be deposited on and/or uniformly distributed within the pore system of the porous molded body, which may then modify the pore structure and result in a substantially homogeneous pore size and pore distribution. The above-mentioned processes may be carried out at temperatures of about 500 to 2000° C., or preferably about 500 to 1500° C., or more preferably about 700 to 1300° C.,

Pores in the carbon-containing porous molded body can be reduced in size by using CVD processes to promote partial or complete closure of the pores. The adsorptive and/or mechanical properties of the molded body can thus be adjusted in a controlled manner.

The carbon-containing porous molded body can be modified by carbide or oxycarbide formation, for example in an oxidation-resistant fashion, by CVD of silanes or siloxanes mixed with hydrocarbons.

In certain exemplary embodiments of the present invention, the porous molded bodies can be coated or modified by means of sputtering. Suitable sputter targets may comprise carbon, silicon, metals or metal compounds, and conventional sputtering methods may be used to deposit these materials onto the porous body. Materials that can be sputtered include, but are not limited to, Ti, Zr, Ta, W, Mo, Cr or Cu, which can be deposited by sputtering onto or into the porous molded bodies, where the corresponding carbides may be formed.

Furthermore, the surface properties of the porous molded body can be modified by means of ion implantation. Nitride, carbonitride or oxynitride phases with incorporated transition metals can be formed by ion implantation of nitrogen, which can significantly increase the chemical resistance and mechanical resistivity of the carbon-containing porous molded body.

Coating with, for example, liquid-crystal tar pitch can result in asymmetric material properties depending on the alignment of the lattice structures during the subsequent cross-linking, carbonization or graphitization. The resulting asymmetrical properties may include, for example, thermal expansivity, mechanical properties, electrical conductivity, and the like.

In certain exemplary embodiments of the present invention, the porous molded bodies may be at least partially coated with a coating of biologically degradable or resorbable polymers such as, e.g., collagen, albumin, gelatin, hyaluronic acid, starch, celluloses such as methyl cellulose, hydroxypropyl cellulose, hydroxypropylmethyl cellulose, carboxymethyl cellulose phthalate; casein, dextrans, polysaccharide, fibrinogen, poly(D,L-lactide), poly(D,L-lactide-co-glycolide), poly(glycolide), poly(hydroxybutylate), poly(alkylcarbonate), poly(orthoester), polyester, poly(hydroxyvaleric acid), polydioxanone, poly(ethylene terephthalate), poly(malic acid), poly(tartaric acid), polyanhydride, polyphosphazene, poly(amino acids) and their co-polymers, or non-biologically degradable or resorbable polymers. Anionic, cationic or amphoteric coatings may also be used such as, for example, alginate, carrageenan, carboxymethyl cellulose; chitosan, poly-L-lysine, or phosphorylcholine.

After carbonization and/or after optionally performing after-treatment procedures, the porous molded body can be subjected to further chemical or physical surface modifications. Cleaning steps can be performed to remove any residue or impurities. Certain acids, including oxidizing acids, or solvents can be used for this purpose, and impurities may be removed by boiling them out in acids or solvents.

The pH and the buffer capacity in an aqueous environment of the molded bodies can be adjusted over wide ranges by a suitable choice of initial substances and additives. The pH in water of the molded bodies can be in the range of about 0 to 14, preferably in the range of about 6 to 8 and more preferably about 6.5 to 7.5. The buffer range of the molded bodies produced may lies in the neutral to acidic range, or preferably in the weakly acidic range. The buffer capacity can be up to about 50 mol/liter, preferably up to about 10 mol/liter, or more preferably about 0.5 to 5 mol/liter.

Exemplary Molded Bodies

The molded bodies produced in accordance with exemplary embodiments of the present invention may be produced in various two- or three-dimensional shapes. For this purpose, the semi-finished molded parts can be processed from the organic polymer materials, optionally mixed with polymeric or non-polymeric fillers, and suitably shaped to produce blanks that can correspond to the final shapes of the porous carbon-based molded bodies, allowing for dimensional shrinkage which may occur during carbonization. The porous molded bodies can be produced in the form of tubes, round rods, plates, blocks, rectangular parallelepipeds, cubes, solid or hollow spheres, flanges, seals, housings, and the like, or they can have an elongated shape such as, for example, round columns, polygonal columns including triangular columns or bars, or plates. The molded body shapes may also be polygonal, such as tetrahedral, pyramidal, octahedral, dodecahedral, icosahedral, rhomboidal or prismatic. Alternatively, they may be in a shape of a sphere or a ball, a spherical or cylindrical lens or annulus, or a honeycomb. The exemplary molded bodies may comprise straight or curved channels which can be wound or folded with different channel diameters and directions (i.e., the channels may be parallel, perpendicular, or they may have arbitrary angles between them).

In a certain exemplary embodiment of the present invention, a tube of porous carbon-based material may be produced. Such an exemplary tube may be formed by carbonizing a carbon-containing molded body comprising a hose of natural or synthetic rubber or suitable plastic, which may optionally be reinforced with fiber or fabric inserts. A textile fabric impregnated with synthetic resins and shaped in the form of a hose may also be used as a semi-finished molded part to produce a tube of porous carbon-based material by subsequent carbonization.

A hose used to produce a porous tube can have a multilayer structure, for example, it may comprise an inner layer of foamed plastic and an outer layer of non-foamed plastic, or vice-versa. The hose may also comprise more than two layers.

The multilayer hose may also be produced as a semi-finished molded part by co-extrusion blow molding, and it may then be carbonized to form a tube of porous carbon-based material.

In a further exemplary embodiment of the present invention, a tube of carbon-based material can be produced by winding a paper material impregnated or coated with polymer materials to form a tube, where the winding can be performed, for example, on a lathe or around a core or mandrel, whereby the tube can then be carbonized under carbonization conditions to form a porous carbon-containing tube. The tube may comprise a flat fiber fabric, channel structures or felt structures, or combinations thereof, which can be impregnated and/or coated with organic polymer materials and then wound around a core or by means of a suitable mandrel. The mandrel or core can be optionally removed before or after the carbonization procedure. In this way, porous tubes can be produced which may further be after-treated, post-compacted or sealed as described above. Porous tubes thus produced can be completely or partially sealed by suitable after-treatment steps such as, for example, CVD or coating using, e.g., organic polymers.

Semi-finished molded parts may also comprise tubes such as long polymer hoses, which can be used to produce continuous carbon tubes. Fiber-reinforced hoses may also be used, where the fibers can include, e.g., textile or fabric fibers, glass fibers, carbon fibers, rock wool, polymer fibers such as those made from polyacrylnitrile, nonwoven materials, fiber nonwovens, felts, cellulose, PET fibers, or any mixtures of these materials.

Asymmetric structures of carbon-containing molded bodies can be produced by using multilayer semi-finished molded parts. For example, foamed polymer materials such as polyurethane foam, polyacrylnitrile foam and the like can be molded with an additional layer of dense polymer material, which can then be carbonized to form molded bodies having a porosity distribution that varies in different regions of the body.

Flanges can be laminated onto semi-finished molded parts comprising hollow bodies, which may then be through-carbonized with closed pores. When using polymer fibers or fabrics, solid-carbon module units exhibiting exceptional adhesion between fiber and matrix may be produced.

The carbon-based molded bodies, including carbon tubes, can be used as tubular membranes, in tube membrane reactors, in tube bundle reactors and heat exchangers, and also in bioreactors. The molded bodies can also be used as porous catalyst supports, especially in the automobile field, or they may be used for flue-gas purification in technical installations. Certain advantages of these carbon-based molded bodies over other materials may include their heat resistance, chemical resistance, and dimensional stability. Furthermore, the molded bodies and materials can be almost free of stress and extremely stable under thermal shock, i.e., severe jumps in temperature may be tolerated without introducing mechanical or structural problems. In certain exemplary embodiments of the present invention, long-term stable and highly effective catalyst supports can be produced by incorporating metals, including precious metals and other catalytically active materials, into the molded bodies.

Plates comprising flat channel structures or tube structures wound from them may be suitable for use as insulating materials, e.g., for high-temperature applications or for shielding microwaves (e.g., a microwave absorber). The electrical properties may be adjusted so that, for example, high-frequency heaters can couple their energy into the furnace area through these insulating materials almost free from losses. Alternatively, properties of highly oriented materials may be adjusted so that the materials are directly excited by high frequency energy and thus can be directly heated. This property may also be used to facilitate carbonization or graphitization of the molded bodies.

Molded bodies can also be used as medical implants, for example, orthopedic, surgical and/or non-orthopedic implants such as bone or joint prostheses, orthopedic plates, screws, nails, and the like.

In certain exemplary embodiments of the present invention, molded bodies can be used as substrates or carriers for colonization by micro-organisms or cell cultures based on their biocompatibility and robust surface properties such as, e.g., adsorption capacity, absorptive capacity, adhesion of biological material, adjustable porosity, pore sizes and volumes, including closed-pore molded bodies, and the like.

Carbon-based, carbon-containing molded bodies produced according to the exemplary methods according to the present invention described above, including ceramic materials and composites, may be used as carrier and/or culture systems (TAS) for the cultivation of primary cell cultures such as eukaryotic tissue, e.g. bone, cartilage, liver, kidneys, as well as for the cultivation or immobilization of xenogenic, allogenic, syngenic or autologous cells and cell types, or optionally of genetically modified cell lines.

In addition to the molded bodies produced in accordance with the exemplary methods of the present invention described above, other porous or non-porous carbon-containing materials may be suitable for use as carrier and culture systems (TAS) for the cultivation of primary cell cultures. In addition to the molded bodies described herein, other materials may be used such as those described in International Patent Publication WO 02/32558. Textured carbon- or ceramic-based materials, which may be symmetrical or asymmetrical, or combinations thereof may also be suitable for use as carrier and culture systems.

Molded bodies can be used as carrier and culture systems for nerve tissue. Carbon-containing materials may be particularly suitable and adaptable for cultivation of nerve tissue, through adjustment of the conductivity of the molded body and the application of pulsed currents.

Molded bodies used as carrier and culture systems may also be used as in vitro or in vivo guide structures, i.e., “scaffolds” for two- and three-dimensional tissue growth. Because the molded bodies can be specifically shaped, they may be used to cultivate organ parts or entire organs from cell cultures. In such exemplary applications, the carrier and culture systems may support or modulate cell, tissue or organ growth as guide structures by suitable adjustment of the porosity, by flow-channel design, and by the two- or three-dimensional shaping of the molded bodies, and further by optionally adjusting the provision, distribution and/or replenishment of nutrient solution or medium at the usage site, and further by optionally supporting or promoting cell and tissue proliferation and differentiation.

Materials and molded bodies used as carrier and culture systems can be shaped in two and/or three dimensions. Suitable macrostructures include, for example, tubes, e.g. for the production or cultivation of natural vessels, cubic forms, or other shapes as described above.

The molded bodies may be provided in the shape of natural organs, e.g., cartilaginous joint surfaces of knee, hip, shoulder, finger joints etc., and may further be used as carrier and culture systems to culture suitably shaped cartilage, periosteum and the like. They can then be implanted with the grown tissue, or the cultured tissue can be separated in suitably grown form by conventional methods such as, for example, mechanical or chemical enzymatic detachment, and then implanted.

Carbon-based molded bodies can also exhibit good mechanical properties, which may allow them to be used as implants, e.g., as artificial joints and the like. They can be used as substrates or carriers for a tissue culture and, following the growth of a sufficient layer of cartilage, they can then be used as highly compatible biomimetic implants in the body of patients. Thus, individual patient implants can be provided which are coated with the body's own tissue grown directly on the implant from the patient's own cell samples. This can reduce or eliminate rejection phenomena and immune defense reactions to such implants.

In exemplary embodiments of the present invention, the molded bodies and materials may be used as carrier and culture systems for cultivation in existing bioreactor systems, e.g., passive systems that do not have continuous control technology such as tissue plates, tissue bottles, roller bottles and the like. They can also be used in active systems that may comprise a gas supply and/or automatic adjustment of parameters (acidity, temperature), or other forms of active measurement and control.

Carrier and culture systems based on porous molded bodies as described above may be operated as reactor systems, optionally in modular fashion in corresponding series reactor systems and tissue cultures, wherein suitable devices such as, for example, connections for perfusion with nutrient solutions and gas exchange may be provided.

Carrier and culture systems based on porous molded bodies as described above may also be used as ex vivo reactor systems, e.g., extracorporeal assistance systems, or as organ reactors, e.g., so-called liver assist systems or liver replacement systems; or also in vivo or in vitro for encapsulated islet cells such as an artificial pancreas, encapsulated urothelial cells, e.g. as an artificial kidney and the like, where such exemplary carrier and culture systems may further be implantable.

Carrier and culture systems based on the exemplary porous molded bodies as described above may also be modified to promote organogenesis, for example, with proteoglycans, collagens, tissue-type salts, e.g. hydroxylapatite and the like, including those comprising biologically degradable or resorbable polymers as described above.

Carrier and culture systems based on porous molded bodies as described above may also be modified by impregnation and/or adsorption of, for example, growth factors, cytokines, interferons, and/or adhesion factors. Examples of suitable growth factors may include, but are not limited to, PDGF, EGF, TGF-α, FGF, NGF, erythropoietin, TGF-β, IGF-I or IGF-II. Suitable cytokines may include, for example, IL-1-α or -β, IL-2, IL-3, IL-4, IL-5, IL-6, IL-7, IL-8, IL-9, IL-10, IL-11, IL-12, or IL-13. Suitable interferons may include, for example, INF-α or -β, INF-γ. Examples of suitable adhesion factors may include fibronectin, laminin, vibronectin, fetuin, poly-D-lysin and the like.

Carrier and culture systems based on porous molded bodies as described above may also be used as microarray systems for drug discovery, tissue screening, tissue engineering, and the like.

### EXAMPLES

The following examples are provided to illustrate certain embodiments of the present invention, and are not intended to be restrictive or to limit the scope of the exemplary embodiments of the present invention as described herein.

### Example 1

A tube 500 mm long with a 300 mm wall thickness was produced by winding a glass fiber fabric of E-CR-glass (chemical-resistant modified E glass), 30 mm wide, that was coated/impregnated with phenol-resin-based GFK resin, where the fiber fabric was laid crosswise on a suitable steel mandrel and the mandrel then removed. The weight of the tube was 3.6 g/cm before pyrolysis. Pyrolysis was carried out in a nitrogen atmosphere at 800° C. for 48 hours. The weight of the tube after pyrolysis was 3.0 g/cm. The membrane properties were measured using the bubble-point test (ASTM E1294), and a pore size of 500 Å was observed.

### Example 2

A tube was produced by winding as described in Example 1 above, using a glass fiber nonwoven of C-glass (chemical-resistant C glass, nonwoven), 30 mm wide, and vinyl-ester-resin-based GFK resin, where the glass fiber nonwoven was laid cross-wise on a steel mandrel. The weight of the tube was 3.5 g/cm before pyrolysis. Pyrolysis was carried out in a nitrogen atmosphere at 800° C. for 48 hours. The weight of the tube after pyrolysis was 0.9 g/cm. The membrane properties were measured using the bubble-point test (ASTM E1294), and a pore size of 0.8 micron was observed.

### Example 3

A tube was produced by winding as described in Example 1 above, using a polyacrylnitrile (PAN) nonwoven (Freudenberg), 30 mm wide, and phenol-resin-based GFK resin, where the glass fiber nonwoven was laid cross-wise on a steel mandrel. The weight of the tube was 3.5 g/cm before pyrolysis. Pyrolysis was performed in a nitrogen atmosphere at 800° C. for 48 hours. The weight of the tube after pyrolysis was 1.94 g/cm. The membrane properties were measured using the bubble-point test (ASTM E1294). No pore size (gas breakthrough) was obserevd in the measurement range. Subsequent partial oxidation in an air flow at 400° C. for 15 minutes yielded an average pore size of 1.2 μm, as indicated by the bubble-point test.

### Example 4

A tube was produced by winding as described in Example 1 above, using a glass-fiber nonwoven of E-CR glass (chemical-resistant modified E glass), 30 mm wide, and polyacrylnitrile (PAN) nonwoven (Freudenberg), 30 mm wide (at a 1:1 ratio of the two nonwovens), and phenol-resin-based GFK resin, where the glass fiber nonwovens were laid cross-wise on a steel mandrel. The weight of the tube was 3.6 g/cm before pyrolysis. Pyrolysis was performed in a nitrogen atmosphere at 800° C. for 48 hours. The weight of the tube after pyrolysis was 2.0 g/cm.

### Example 5

A tube was produced by winding as described in Example 1 above, using a glass-fiber nonwoven of E-CR glass (chemical-resistant modified E glass), 30 mm wide, and polyacrylnitrile (PAN) nonwoven (Freudenberg), 30 mm wide (at a 1:1 ratio of the two nonwovens), and phenol-resin-based GFK resin with 20% Aerosil R972, where the glass fiber nonwovens were laid cross-wise on a steel mandrel. The weight of the tube was 3.6 g/cm before pyrolysis. Pyrolysis was performed in a nitrogen atmosphere at 800° C. for 48 hours. The weight of the tube after pyrolysis was 3.0 g/cm.

The Aerosil was then washed out using a 30% NaOH alkali solution. The membrane properties were measured using the bubble-point test (ASTM E1294), and a pore size of 0.6 μm was observed.

### Example 6

Carbon-based plates of natural-fiber-reinforced composite polymer with inorganic fillers were produced, having a weight per unit area of 100 g/m² and a thickness of 110 micron. This flat composite material was provided with a channel structure by a commercially available embossing machine, which yielded a channel diameter of 3 mm after placing one sheet on top of the other. These sheets were glued to form honeycomb-shaped blocks and were then carbonized in a protective gas atmosphere (nitrogen) at 800° C. for 48 hours. The pressure loss in the channel direction was only 0.1 bar/m and a weight loss of 66 wt. % was observed during carbonization.

A tube was then wound from this material, 10 cm long and 40 mm in diameter, with a wall thickness of 6 mm. The tube was adjusted in a coupling-in test in a 8 kHz high-frequency heating device. The current showed almost no variation relative to the quiescent current, and after 5 minutes no significant heating of the material was observed. The material thus produced could be easily sawn, drilled, or milled precisely with no apparent structural or shaping problems.

### Example 7

A natural-fiber-containing polymer composite having a weight per unit area of 100 g/m² and a thickness of 110 μm, was carbonized in a nitrogen atmosphere at 800° C. for 48 hours to form a carrier material for cell culture systems. Air was added towards the end of the carbonization process to modify the pores. A weight loss of 50 wt. % was observed. The resulting material has a pH of 7.4 in water and a buffer range of a weak acid. Pieces of this carbon-based material measuring 20×40 mm and 60 μm thick were placed on conventional six-well tissue plates, and each piece was fed with 4 ml of nutrient solution and 1.5 ml of cell suspension. The cell suspension contained hybridoma FLT₂ cell lines producing MAB against shigatoxin, which is known for its non-adherent, non-adhesive suspension-resistant growth.

For comparison, six-well tissue plates were used under similar conditions and loading, but without the carbon-based material.

The samples using the carbon-based material carriers revealed a spontaneous quantitative immobilization of the cells, and no clouding of the suspension was observed. Within an incubation time of 7 days, the cell density was observed to increase sevenfold to 1.8×10⁷ cells per ml. The MAB production increased from initially 50 μg/ml to 350 μl/ml of the average culture lifetime without any signs of proteolytic degradation. All twelve samples were still living after 25 days, after which incubation was interrupted. These results suggest that the carriers result in an interruption of the contact inhibition despite the higher cell density. Even after crycoconservation and thawing, MAB production was spontaneously restored after adding fresh nutrient medium.

In the comparative experiment that did not include the carbon-based material carriers, only one of the six cultures survived until the eleventh day.

Having thus described in detail several exemplary embodiments of the present invention, it is to be understood that the invention described above is not to be limited to particular details set forth in the above description, as many apparent variations thereof are possible without departing from the spirit or scope of the present invention. The embodiments of the present invention are disclosed herein or are obvious from and encompassed by the detailed description. The detailed description is given by way of example, and is not intended to limit the invention solely to the specific embodiments described.

The foregoing applications, and all documents cited therein or during their prosecution (“appln. cited documents”) and all documents cited or referenced in the appln. cited documents, and all documents or other publications cited or referenced herein (“herein cited documents”), and all documents or publications cited or referenced in the herein cited documents, together with any manufacturer's instructions, descriptions, product specifications, and product sheets for any products mentioned herein or in any document incorporated by reference herein, are hereby incorporated herein by reference, and may be employed in the practice of the invention. Citation or identification of any document in this application is not an admission that such document is available as prior art to the present invention.

It is noted that in this disclosure and particularly in the claims, terms such as “comprises,” “comprised,” “comprising” and the like can have the meaning attributed to them in U.S. Patent law; e.g., they can mean “includes,” “included,” “including” and the like; and that terms such as “consisting essentially of” and “consists essentially of” can have the meaning ascribed to them in U.S. Patent law, e.g., they allow for elements not explicitly recited, but exclude elements that are found in the prior art or that affect a basic or novel characteristic of the invention.

What is claimed is:

1. A method for producing porous molded bodies comprising:
(a) forming at least one first molded part from a composition comprising at least one organic polymer material;
(b) carbonizing the at least one first molded part in a non-oxidizing atmosphere to form at least one second molded part; and
(c) producing at least one molded body with pores from the at least one second molded part.

2. The method of claim 1, wherein the composition further comprises at least one polymeric filler, and wherein steps (b) and (c) are performed simultaneously, and wherein the at least one polymeric filler is at least substantially decomposed.

3. The method of claim 2, wherein the at least one polymeric filler comprises at least one of saturated aliphatic hydrocarbon homopolymers, branched aliphatic hydrocarbon homopolymers unbranched aliphatic hydrocarbon homopolymers, saturated aliphatic hydrocarbon copolymers, branched aliphatic hydrocarbon copolymers unbranched aliphatic hydrocarbon copolymers, polyolefins, polyethylene, polypropylene, polybutene, polyisobutene, or polypentene.

4. The method of claim 1, wherein the at least one organic polymer material comprises at least one of unsaturated branched aliphatic hydrocarbons, branched cross-linked aromatic hydrocarbons, unbranched cross-linked aromatic hydrocarbons, branched non-cross-linked aromatic hydrocarbons, unbranched non-cross-linked aromatic hydrocarbons, branched cross-linked partially aromatic hydrocarbons, unbranched cross-linked partially aromatic hydrocarbons, branched non-cross-linked partially aromatic hydrocarbons, unbranched non-cross-linked partially aromatic hydrocarbons, or substituted derivatives of any of the preceding.

5. The method of claim 1, further comprising treating the at least one molded body with at least one of an oxidizing agent or a reducing agent.

6. The method of claim 1, wherein step (c) comprises partially oxidizing the at least one second molded part to produce pores therein.

7. The method of claim 6, wherein step (c) further comprises heat treating the at least one second molded part in an oxidizing gas atmosphere.

8. The method of claim 7, wherein the oxidizing gas atmosphere comprises at least one of air, oxygen, carbon monoxide, carbon dioxide or a nitrogen oxide, and wherein step (c) is performed at temperatures in the range of about 50 to 800° C.

9. The method of claim 6, wherein step (c) comprises exposing the at least one second molded part to oxidizing acids.

10. The method of claim 1, wherein the at least one organic polymer material comprises at least one of polybutadiene, a polyvinyl, polyvinylchloride, polyvinyl alcohol, poly(meth)acrylic acid, polyacryl cyanoacrylate, polyacrylnitrile, polyamide, polyester, polyurethane, polystyrene, polytetrafluoroethylene, collagen, albumin, gelatin, hyaluronic acid, starch, cellulose, methylcellulose, hydroxypropylmethyl cellulose, carboxymethyl cellulose phthalate, casein, dextran, polysaccharide fibrinogen, poly(D,L-lactide), poly(D,L-lactide-co-glycolide), polyglycolide, polyhydroxybutylate, polyalkylcarbonate, polyorthoester, polyester, polyhydroxyvaleric acid, polydioxanone, polyethylene terephthalate, polymalic acid, polytartaric acid, polyanhydride, polyphosphazene, polyamino acids; polyethylenevinyl acetate, silicone; poly(ester urethane), poly(ether urethane), poly(ester urea), a polyether, polyethylene oxide, polypropylene oxide, pluronics, polytetramethylene glycol, polyvinyl pyrrolidone, poly(vinyl acetate phthalate), alkyd resin, chlororubber, epoxy resin, acrylate resin, phenol resin, amine resin, melamine resin, an alkylphenol resin, an epoxided aromatic resin, tar, a tar-like material, tar pitch, a liquid-crystal tar pitch, bitumen, starch, cellulose, shellac, polyacrylnitrile fibers, cellulose fibers, novolak fibers, organic materials of renewable raw materials, or copolymers of any of the preceding.

11. The method of claim 1, wherein the composition further comprises at least one of a filler, a softener, a lubricant, a flame retardant, glass, glass fibers, carbon fibers, cotton, fabric, metal powder, metal compounds, metal oxides, silicon, silicon oxide, zeolites, TiO₂, aluminium oxide, aluminosilicate, zirconium oxide, talc, graphite, soot, clay materials, or phyllosilicates.

12. The method of claim 1, wherein step (a) comprises at least one of casting, extruding, pressing, embossing, or injection molding the composition.

13. The method of claim 1, wherein step (b) is carried out under a protective gas atmosphere.

14. The method of claim 13, wherein the protective gas atmosphere comprises at least one of nitrogen or argon.

15. The method of claim 13, wherein the protective gas atmosphere comprises a reactive gas.

16. The method of claim 15, wherein the reactive gas is hydrogen.

17. The method of claim 1, wherein step (b) is performed at temperatures in the range of about 200 to 4000° C.

18. A porous molded body produced by:
(a) forming at least one first molded part from a composition comprising at least one organic polymer material;
(b) carbonizing the at least one first molded part in a non-oxidizing atmosphere to form at least one second molded part; and
(c) producing the molded body with pores from the at least one second molded part.

19. The molded body of claim 18, wherein the molded body is in the shape of at least one of a tube, a round rod, a plate, a block, a rectangular parallelepiped, a cube, an injection mold, a honeycomb structure, an imprinted structure, a folded structure, a wound structure, a rolled two-dimensional structure, a rolled three-dimensional structure, a channeled structure, a solid sphere, a hollow sphere, a flange, a seal, or a housing.

20. The molded body of claim 18, wherein the molded body is configured to be used as at least one of a carrier or a culture system for the cultivation of primary cell cultures.

21. The molded body of claim 20, wherein the cell cultures comprise at least one of eukaryotic tissue, bone, cartilage, liver cells, kidney cells, pancreas cells, nerves, xenogenic cells, allogenic cells, syngenic cells, autologous cells, or genetically modified cell lines.

22. The molded body of claim 21, wherein the molded body is configured to be used as a guide structure for growth of tissue.

23. The molded body of claim 22, wherein the tissue comprises at least part of a bodily organ.

24. The molded body of claim 19, wherein the molded body is configured to be used as an ex vivo reactor system.

25. The molded body of claim 20, wherein the molded body is configured to be used in vivo as an implant.

26. The molded body of claim 20, wherein the molded body is modified with at least one of proteoglycans, collagens, tissue-type salts, growth factors, biologically degradable polymers, or resorbable polymers.
